(19) United States
(12) Patent Application Publication
(10) Pub. No.: US 2009/0012039 A1
(43) Pub. Date: Jan. 8, 2009

(54) Use of methylcobalamin nasal spray to treat disorders

(76) Inventor: Stan Kurtz, Tarzana, CA (US)

Correspondence Address: FULBRIGHT AND JAWORSKI LLP, 555 S. FLOWER STREET, 41ST FLOOR, LOS ANGELES, CA, 90071, US

(21) Appl. No.: 12/077,296

(22) Filed: Mar. 17, 2008

Related U.S. Application Data
(60) Provisional application No. 60/918,526, filed on Mar. 16, 2007.

(51) Int. Cl. A61K 31/714; A61P 25/00; A61P 1/00; A61P 31/12; A61P 29/00; A61P 37/00

(52) U.S. Cl. 514/52

(57) ABSTRACT

A method of treating a disorder by nasally administering methylcobalamin, with or without folinic acid. The disorders addressed are: a) attention deficit hyperactivity disorder (ADHD); b) anxiety, depression, stress and chronic stress; c) socialization problems, mood problems, behavior problems, memory problems; d) dislexia, depth perception problems, color viewing problems, visual and auditory processing problems, light modulation problems, night vision problems; e) speech problems such as finding words, apraxia, and articulation problems, sleep regulation problems, eye or muscle movement problems; and f) chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, autoimmune problems. In some embodiments, the disorders that are particularly addressed are ADHD, anxiety, stress and chronic stress, and irritable bowel.

### REFERENCE TO PRIOR APPLICATIONS

This application claims the benefit of U.S. Provisional Application No. 60/918,526, filed on Mar. 16, 2007, which is incorporated by reference herein.

### BACKGROUND

1. Field of Invention

This disclosure relates generally to nasal administration of methylcobalamin for treatment of psychological and neurophysiological disorders.

2. Related Art

Attention deficit hyperactivity disorder (“ADHD’), also called attention deficit disorder (“ADD”), is a neurobehavioral condition of children and adults that is characterized by a chronic level of inattention, hyperactivity, and impulsivity. Three types of ADHD are recognized. In the predominantly inattentive type, a person can have six or more of the following disruptive and age-inappropriate symptoms: difficulty paying attention to details, difficulty keeping attention on tasks, difficulty following instructions, difficulty organizing activities, difficulty following conversations, easily distracted, and forgetful of daily routines. In the predominantly hyperactive-impulsive type, a person can have six or more of the following disruptive and age-inappropriate symptoms: fidgeting often, inappropriate running about, trouble playing or enjoying leisure activities quietly, excessive talking, blurting out answers, trouble waiting turn, and interrupting others. In the combined type, both inattentive and hyperactive-impulsive behaviors can be present. The Diagnostic & Statistical Manual for Mental Disorders (DSM-IV-TR), 2000, provides various criteria for diagnosing ADHD disorders.

Irritable bowel, or irritable bowel syndrome, is a disease characterized by abdominal cramping, bloating, flatulence, chronic constipation and/or diarrhea, and mucus in the stool. Anxiety is a disorder characterized by persistent, irrational fear or worry. Examples of anxiety disorders include post-traumatic stress disorder and phobias such as irrational fear of spiders or open spaces. Chronic stress is an ongoing psychological and physiological state, also known as the “fight or flight” response, to various stimuli. Symptoms of chronic stress include cognitive, emotional, behavioral and physical problems.

Methylcobalamin is a vitamin B12 derivative that mediates methyl group transfer in the metabolic generation of methionine from homocysteine. Like vitamin B12, methylcobalamin is based on a corrin ring and has the formula C₆₃H₉₁CoN₁₃O₁₄P.

Autism is a developmental disorder characterized by impaired social interaction, repetitive or severely limited activities and interests, and verbal and nonverbal communication problems. Based on studies of oxidative stress and methionine synthase, subcutaneous injection of methylcobalamin has been found to be helpful in children with autism. Studies on the intranasal spray of hydroxycobalamin, a different vitamin B12 derivative, have suggested that this route of administration is similar in efficacy to intravenous administration of hydroxycobalamin, and that intranasal spray of hydroxycobalamin can provide a cobalamin normalizing effect that other routes of administration do not seem to have.

Methylcobalamin, not administered nasally, has been reported to help improve oxidative status in children with autism, and is suggested to be an antiviral for AIDS, to be helpful in fighting cancer, peripheral neuropathy, and autonomic disregulation, and to help improve DNA methylation and nerve regeneration. The present inventor has discovered that intranasal administration of methylcobalamin can be an effective method of treatment for ADHD and other illnesses and disorders, as herein described.

### SUMMARY OF THE DISCLOSURE

In one aspect, a method of treating a person having a psychological or neurophysiological disorder is provided. The method comprises nasally administering methylcobalamin, or a pharmaceutically acceptable salt thereof, to a person in need of such treatment in an amount sufficient to treat the disorder in the person. The disorder can be: a) ADHD; b) anxiety, depression, stress and chronic stress; c) socialization problems, mood problems, behavior problems, memory problems; d) dislexia, depth perception problems, color viewing problems, visual and auditory processing problems, light modulation problems, night vision problems; e) speech problems such as finding words, apraxia, and articulation problems, sleep regulation problems, eye or muscle movement problems; and f) chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, autoimmune problems.

In certain embodiments, the disorders that are particularly addressed are ADHD, anxiety, stress and chronic stress, and irritable bowel.

In various embodiments, the treatment reduces one or more symptoms or characteristics of the particular disorder. For example, when the disorder is ADHD, the treatment can reduce hyperactivity symptoms of ADHD and/or inattentiveness symptoms of ADHD. The methylcobalamin, or pharmaceutically acceptable salt thereof, can be administered with or without folinic acid in various embodiments.

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a topograph showing a subject's theta wave activity before nasal administration of methylcobalamin.

FIG. 2 is a topograph showing a subject's theta wave activity after nasal administration of methylcobalamin.

FIG. 3A-D is a panel of topographs showing a subject's theta wave activity before (A) and after (B-D) nasal administration of methylcobalamin.

FIG. 4A-D is a panel of SPECT scans taken before (A,C) and after (B,D) nasal administration of methylcobalamin.

### DETAILED DESCRIPTION

A method of treating certain psychological or neurophysiological disorders is provided. In various embodiments, the method comprises nasally administering methylcobalamin, or a pharmaceutically acceptable salt thereof, in an amount sufficient to treat one of the disorders in the person. As used herein, the term “disorder” broadly refers to a syndrome, condition, chronic illness or particular disorder. The term “treat” means to reduce one or more symptoms or characteristics of the disorder. For example, symptoms of hyperactivity or inattentiveness in ADHD can be reduced. By “reduce” is meant decreasing the number of events or the severity of a symptom or characteristic. The term “therapeutically effective amount” means an amount sufficient to reduce one or more symptoms or characteristics of ADHD or other disorder.

The disorders can be any of the following: a) ADHD; b) anxiety, depression, stress and chronic stress; c) socialization problems, mood problems, behavior problems, memory problems; d) dislexia, depth perception problems, color viewing problems, visual and auditory processing problems, light modulation problems, night vision problems; e) speech problems such as finding words, apraxia, and articulation problems, sleep regulation problems, eye or muscle movement problems; and f) chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, autoimmune problems. In some embodiments, the disorders that are particularly addressed are ADHD, anxiety, stress and chronic stress, and irritable bowel.

In addition to reducing symptoms or characteristics of the various disorders listed herein, nasal administration of methylcobalamin can also assist in nerve growth, improve the ability to process sounds and modulate background noise, block the effects of alcohol, or reduce the desire for alcohol, coffee, marijuana, methamphetamines, and other addictive drugs and behaviors. Nasal administration of methylcobalamin can also lead to improved brain synchronicity, improved brain blood flow, or reduced post-anesthetic recovery times and symptoms. Some additional symptoms that may be treated or reduced by methylcobalamin nasal administration include white coated tongue, ocular stress (eye “twittering”), night vision challenges, food sensitivities, throat infections, trouble sleeping or waking up in the morning, and difficulty maintaining weight. Eye “twittering” is a condition in which a person has difficulty keeping his or her eyes fixed on one location, and instead frequently moves the eyes from place to place.

Nasal administration of methylcobalamin can also reduce a characteristic associated with a disorder even if there is no formal medical diagnosis of the disorder. For example, nasal administration of methylcobalamin can reduce the pain associated with inflammation and autoimmune responses even though the level of inflammation or autoimmune response is less than the level required for a medical diagnosis. Similarly, nasal administration of methylcobalamin can improve, for example, anxiety, inattentiveness, hyperactivity, impulsivity, vision, and digestion in a subject even though these symptoms are not clearly associated with a medical disorder or diagnosis. Nasal administration of methylcobalamin can also raise energy levels, which can provide benefits to a subject whether or not diagnosed with a disorder.

In a person responsive to methylcobalamin, the effects of methylcobalmin administered nasally can begin to occur within minutes of administration. For example, one experiment on QEEG theta wave activity showed that methylcobalamin nasal spray worked almost instantly. In various experiments, subjects reported feeling better in minutes and sometimes seconds. Thus, improvements can occur within the first hour of administration.

Although the effects of a typical administration can last about 24 hours, every person is different. Thus, some people can become depleted of available methylcobalamin in hours or less, while others can feel the benefits for days. The dosage and frequency of methylcobalamin administration can decrease over time, particularly with changing lifestyle or eating habits. For example, reducing milk and wheat products, lessening complex carbohydrates and starches, and improvements to intestinal flora can lessen the need for methylcobalamin.

It appears that many individuals do not respond to sublingual administration of methylcobalamin. For example, self-reporting by individuals has indicated that only a few people appear to significantly benefit from sublingual methylcobalamin administration. Many anecdotal reports suggest that responders to methylcobalamin administered nasally do not respond to sublingual or oral administration. A deficiency in certain types of bacteria of the intestinal flora and mucosa might have a possible role as well in reducing the effectiveness of sublingual or oral administration.

There appear to be four types of responders to methylcobalamin based on observations of autistic persons: a) people who respond better to nasal administration of methylcobalamin; b) people who respond better to a subcutaneous administration of methylcobalamin; c) people who respond to either mode of administering methylcobalamin; d) people who respond to neither mode of administering methylcobalamin. People with autism appear to respond better to methylcobalmin administered by nasal spray.

In addition, it appears that nasal administration of methylcobalamin can be significantly more effective than sublingual or oral administration of methylcobalamin.

Folinic acid, or a pharmaceutically acceptable salt thereof, can be added as a component of a nasal spray containing methylcobalamin. Folinic acid can aid in the utilization of methylcobalamin, and appears to keep methylcobalamin in the body for a greater period of time. If any hyperactivity or negative symptoms occur, the nasal spray can be tried without folinic acid.

Compositions for nasal administration can, for example, be prepared by dissolving, dispersing, mixing or incorporating methylcobalamin (with or without folinic acid) and optional pharmaceutical adjuvants in an excipient, such as, for example, water, saline, aqueous dextrose, glycerol, ethanol, and the like, to thereby form a solution or suspension. If desired, the composition to be nasally administered can also contain minor amounts of nontoxic auxiliary substances such as wetting or emulsifying agents, pH buffering agents and the like, for example, sodium acetate, sorbitan mono-laurate, triethanolamine sodium acetate, triethanolamine oleate, potassium sorbate, glycerin, lecithin, etc. Actual methods of preparing such dosage forms are known, or will be apparent, to those skilled in this art; for example, see Remington's Pharmaceutical Sciences. Any adjuvant, excipient or auxiliary substance is contemplated so long as it does not prevent the effectiveness of nasally administered methylcobalamin, and preferably, does not irritate nasal passages.

The compositions disclosed herein may be formulated in neutral or salt form. Pharmaceutically-acceptable salts include acid addition salts that are formed with inorganic acids such as, for example, hydrochloric or phosphoric acids, or such organic acids as acetic, oxalic, tartaric, mandelic, and the like. Pharmaceutically-acceptable salts can also be derived from inorganic bases such as, for example, sodium, potassium, ammonium, calcium, or ferric hydroxides, and such organic bases as isopropylamine, trimethylamine, histidine, procaine and the like. The term “pharmaceutically-acceptable” refers to substances and compositions that do not produce an allergic or similar untoward reaction when administered to a human.

Methylcobalamin-containing nasal spray, with or without folinic acid, can be obtained from various “autism friendly” and developmental disorders-focused compounding pharmacies. For example, one such pharmacy to compound methylcobalamin is Coastal Compounding Pharmacy (Savannah, Ga., USA) (on the World Wide Web at coastalcompounding.com). Methylcobalamin nasal spray may require a prescription when acquired in the United States. Doctors who have commonly prescribed methylcobalamin nasal spray for autism and related conditions can be found at the website of the Autism Research Institute (San Diego, Calif., USA), List of Clinicians, (on the World Wide Web at autism.com).

To administer methylcobalamin in adults, in some embodiments about 1250 μg of methylcobalamin together with about 100 μg of folinic acid per about 0.1 ml spray is administered once in each nostril once per day. Methylcobalamin is concentrated at about 12.5 mg/ml and the folinic is at about 1 mg/ml. If pharmaceutically acceptable salts of methylcobalamin and/or folinic acid are used, the amount of these salts is equivalent to the molar amount of methylcobalamin and folinic acid indicated.

In other embodiments, methylcobalamin can be administered from about 500-1500 μg/0.1 ml spray, preferably from about 1000-1250 μg/0.1 ml of spray. In addition, folinic acid can be administered from about 25-300 μg/0.1 ml of spray, preferably from about 25-150 μg/0.1 ml of spray; more preferably from about 25-125 μg/0.1 ml of spray, and even more preferably from about 25-100 μg/0.1 ml of spray. In certain embodiments, methylcobalamin is administered without folinic acid.

In some embodiments for children, about 625 μg of methylcobalamin together with about 50 μg of folinic acid can be administered once per day. If pharmaceutically acceptable salts of methylcobalamin and/or folinic acid are used, the amount of these salts is equivalent to the molar amount of methylcobalamin and folinic acid indicated. Again, in other embodiments, methylcobalamin is administered without folinic acid.

Methylcobalamin is preferably administered after a meal, more preferably after breakfast (since it can provide a feeling of energy throughout the day). For young children who are sensitive to nasal sprays, methylcobalamin can be administered at night while the child is sleeping.

A person can conduct initial trials later in the afternoon when he or she begins to feel tired, which can provide a better idea of the difference pre and post spray.

In other embodiments, the dose can be 1250 μg methylcobalamin and 300 μg of folinic per 0.1 ml of spray. For people sensitive to folinic acid, a lower dosage of folinic acid may be initially used, or methylcobalamin can be used by itself, before increasing folinic dosage.

In the foregoing discussion of dosages, if pharmaceutically acceptable salts of methylcobalamin and/or folinic acid are used, the amount of these salts is equivalent to the molar amount of methylcobalamin and folinic acid indicated.

Due to biochemical, physiological and environmental variations, each person's need for methylcobalamin is different. As little as necessary to obtain the desired results is preferred since using more does not necessarily mean better results. Taking a break from time to time or trying to space out administration can be good practice in determining an appropriate dosage. In addition, greater or lesser dosages than the dosages described above can be appropriate depending on the person.

The following is a suggested regimen for nasal administration of methylcobalamin-containing nasal spray, in a spray bottle. First, the user should prime the bottle before administration. If possible, the user's head should be tilted slightly downward so the angle of the bottle is vertical. Then the nasal spray should be squirted deep into the nasal passage so that little or no methylcobalmin runs out of the nose. The liquid is red like beets or blood, so if some drips out, it may look like blood. Methylcobalmin is very gentle and is unlikely to cause a nosebleed. If the user is subject to nose bleeds (not due to methylcobalamin), the nasal spray can be supplemented with Vitamin K.

In other embodiments, the nasal spray can contain zinc, omega 3 fatty acids (for example, from cod liver oil), other B vitamins such as B6 and pyridoxal 5-phosphate (P5P), B2, and B5, calcium, magnesium, vitamins A, D, and K, taurine or selenium, or any combination thereof.

For a child that is sensitive to sensory stimulus, the spray bottle can be warmed to room temperature before application of the spray. The spray can be administered to a sensitive child at night while the child sleeps.

### EXAMPLES

The present invention may be better understood by referring to the accompanying examples, which are intended for illustration purposes only and should not in any sense be construed as limiting the scope of the invention as defined in the claims appended hereto.

### Example 1

Methylcobalamin nasal spray was shown to normalize brain waves of a 23 year old male with DSM-IV Attention Deficit Disorder. Testing included pre-testing and 3 post tests including one immediately after administration. The tests showed a dramatic lessoning of theta wave activity (3.5-7.5 Hz), which is common in attention challenges, and activity in the entire brain increased, while the subject was observed to be more relaxed and attentive while ocular “twittering” was greatly reduced. QEEG is a brain mapping procedure that records electrical activity within the brain. Theta waves are relatively slow brain waves occurring about 4-7 times per second (3.5-7.5 Hz).

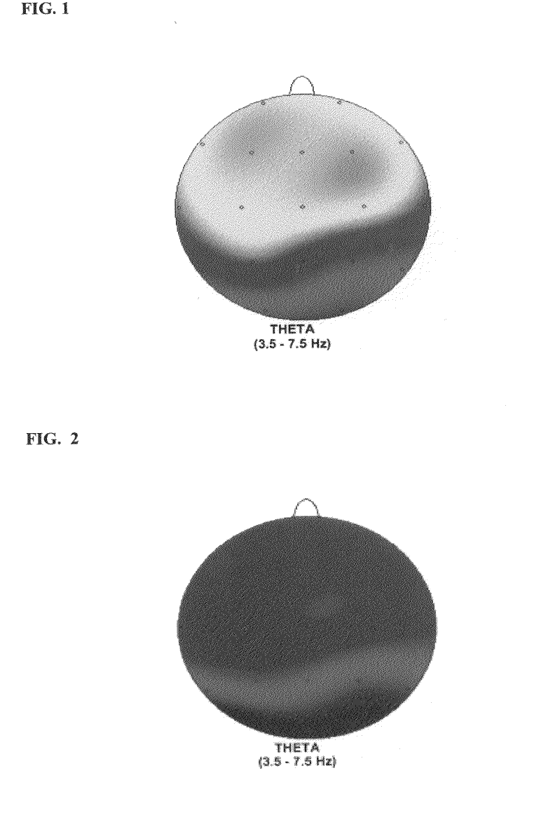

FIG. 1 shows the QEEG theta wave activity before nasal administration of methylcobalamin. FIG. 2 show QEEG theta wave activity after nasal administration of methylcobalamin.

### Further Details

QEEG data was collected from the 23 year old male with DSM IV Attention Deficit Disorder prior to and after treatment of intranasal methylcobalamin and folinic acid. During QEEG setup, the subject showed signs of fatigue including sighing about every 45 seconds for duration of approximately 20 minutes.

The baseline QEEG showed an excessive amount of frontal lobe theta activity (3.5-7.5 Hz) both visually and in computer generated summary analysis. This rhythm was asynchronous with the seemingly normal occipital alpha activity (7.5-12 hz), a common pattern in ADHD. The subject also displayed rapid eye “twittering” movements during the much of the baseline QEEG.

A. Administration

The methylcobalamin/folinic nasal spray was administered in the dose of 1250 μg of methylcobalamin and 300 μg folinic acid in each nostril.

B. Post Administration

The first post reading was taken immediately after the spray and showed a dramatic reduction in frontal theta activity and a reduction in occipital theta activity. The occipital alpha waves were initially higher in amplitude. The subject's eye twittering was greatly reduced. His subjective report was that he was significantly calmer but not particularly more alert.

C. 50 Minutes and 1 Hour, 15 Minutes Post Administration

The readings continued to show less theta activity than baseline and seemingly more synchronized across the brain. There was more alpha activity across the brain and the “background” alpha waves were lowered in amplitude, possibly indicating better access to the CNS. The subject's eyes were progressively more still. His sighing was reduced by 400%. His subjective report was feeling calmer and more alert.

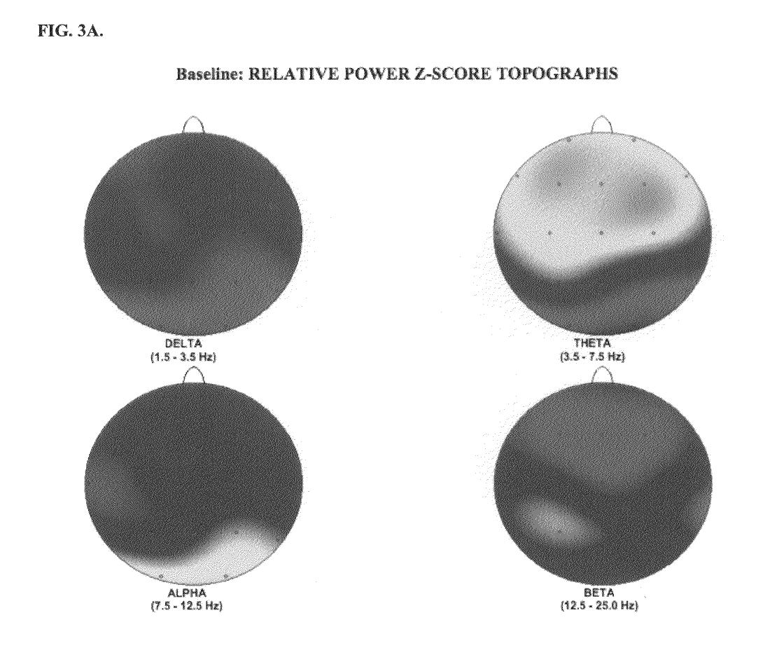

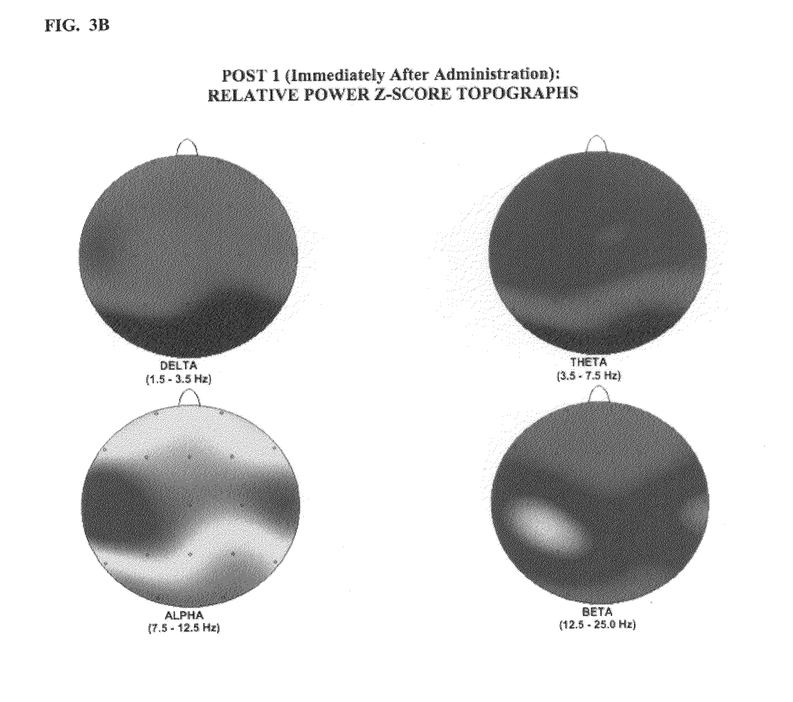

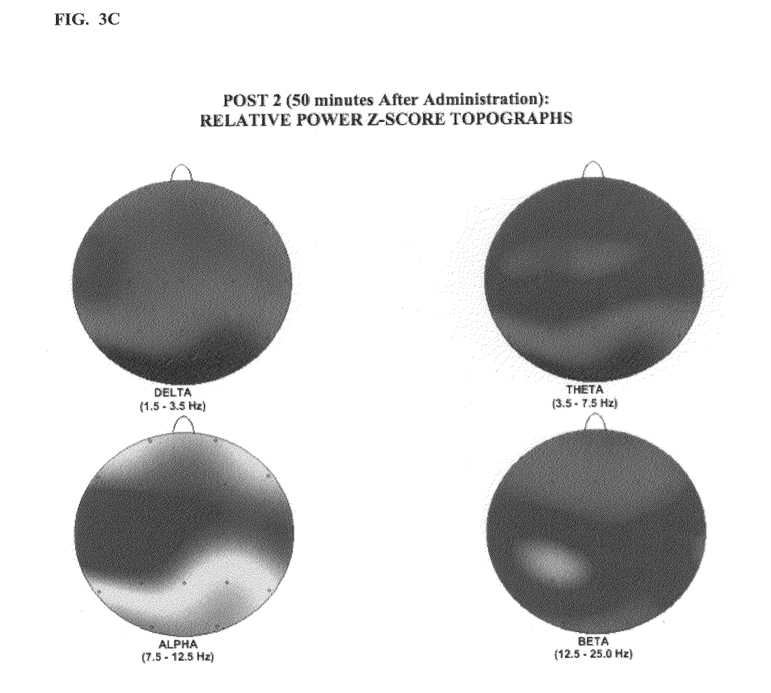

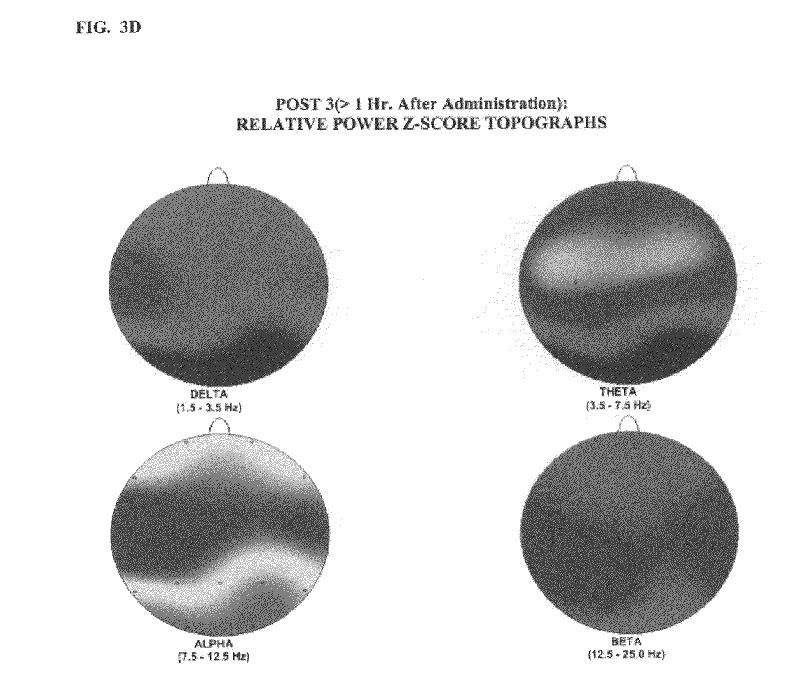

FIG. 3A-D shows topographs for the subject before and after nasal administration of methylcobalamin.

### Example 2

### SPECT Activity

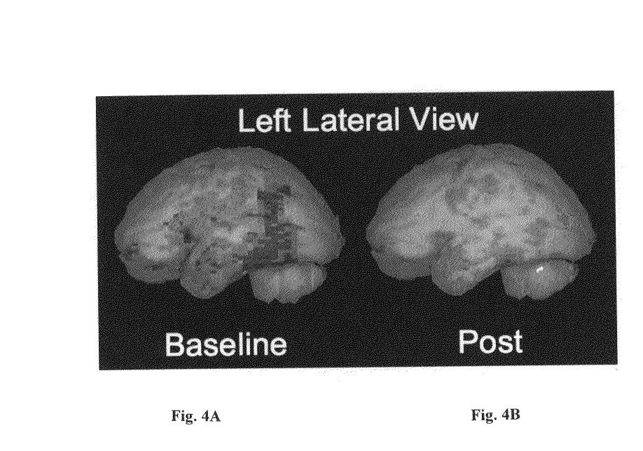

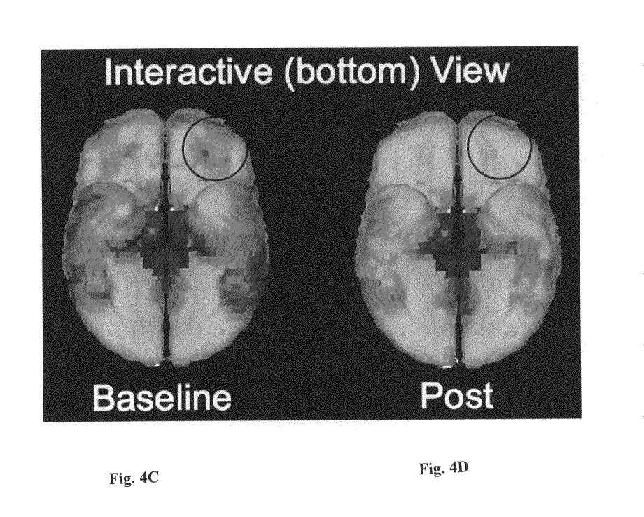

Nasal administration of methylcobalamin was shown to improve brain blood flow of a 53 year old woman with autism who was struggling with fibromyalgia, depression, and symptoms of brain fog, had trouble finding words, had memory issues, attention issues and several other chronic symptoms. Along with other possible abnormalities, the baseline test shows a significant amount of hypoprofusion (reduced blood flow) in the temporal, frontal and prefrontal lobes. This individual was nasally sprayed with methylcobalamin nasal spray 50 minutes prior to SPECT isotope being administered. Scans of the subject post-administration of methylcobalamin are shown in FIG. 4A-D. A SPECT scan is a brain imaging technique that shows brain blood flow and regional brain cell function.

### Example 3

A woman had terrible ADHD and colitis symptoms. She had intestinal surgery and was on Strattera and steroids at the time of this interview. The woman recovered and has been medication free since her trial of methylcobalamin, and she demonstrates a normalization of eye tracking.

A 23-year-old was suffering from irritable bowel and diagnosed with Attention Deficit Disorder. His brain patterns improved in just minutes after administration of methylcobalamin, as measured by QEEG technology.

### Example 5

A woman's symptoms of acid reflux, back pain, post nasal drip, swollen glands, upper respiratory infection and sore throat along with chronic stress and fibromyalgia cleared in minutes after nasal administration of methylcobalamin.

### Example 6

A preschool teacher, had headache every day for as long as she could remember (20+ years). Her pain, stress and worry lifted during her first dose of nasal methylcobalamin.

### Example 7

A woman was suffering for most of her adult life with asthma. She was taking three steroids and still having difficulty. She also had sleep challenges, reading difficulty, eye tracking issues, OCD tendencies, and stress. In minutes she improved on nasal methylcobalamin.

### Example 8

A woman felt different from everyone else. She had difficulty socializing and felt chronically stressed and full of anxiety. She says she didn't feel normal until she tried nasal methylcobalamin.

### Example 9

A woman was suffering from chronic stress, fatigue, asthma, high blood pressure and control issues. She also had eye tracking issues that prevented her from ever reading to her three year old son. According to the woman, a first dose of nasal methylcobalamin changed her life.

### Example 10

A woman was suffering from monocular vision (she could only process data out of one eye). She had no depth perception, had night vision challenges, was basically color blind, had difficulty sleeping and had dyslexia. All of her symptoms were greatly improved with the first dose of methylcobalamin, literally in seconds. She said she has never seen so well in her life.

### Example 11

The symptoms of a 14 year old boy with asthma resolved in minutes. After his first dose of methylcobalamin nasally, he explained how methylcobalamin allowed him to breath like his steroidal inhalers do. This may be an example of how chronic immune system activation may play a role in attention challenges.

### Example 12

A 53 year old woman had been suffering from chronic pain, fatigue, and fibromyalgia for many years. According to the woman, she has became clear and pain free on nasal methylcobalamin. Currently, she maintains her health with diet alone.

### Example 13

A young man had been diagnosed with Chron's and talks about being challenged with anxiety, attention, fatigue, OCD, allergies, breathing difficulties and with studying challenges. Immediately after being nasally sprayed with methylcobalamin, he began to change.

### Example 14

Two sisters, one complains of ADD and the other of OCD and both of them greatly improved with their first dose of nasal methylcobalamin.

### Example 15

A dental assistant had been suffering from chronic fatigue and anxiety since giving birth to a child. She said she improved in seconds after nasal methylcobalamin administration.

### Example 16

A behavior therapist was suffering with chronic anxiety, stress, attention issues, allergies, addictive/impulsive behavior and stomach discomfort. Her symptoms were improved in minutes following nasal methylcobalamin administration.

### Example 17

A woman with a history of drug use says nasal administration of methylcobalamin lessened her cravings for drugs and blocked the effects of alcohol and marijuana.

### Example 18

A young child with autism and speech delay greatly improved during treatment with methylcobalamin nasal spray administration.

What is claimed is:

1. A method of treating a psychological or neurophysiological disorder, comprising nasally administering methylcobalamin, or a pharmaceutically acceptable salt thereof, to a person in need of such treatment in an amount sufficient to treat the disorder in the person, wherein the disorder is selected from the group consisting of:
attention deficit hyperactivity disorder (ADHD), anxiety, depression, stress and chronic stress, socialization problems, mood problems, behavior problems, memory problems, dislexia, depth perception problems, color viewing problems, visual and auditory processing problems, light modulation problems, night vision problems, speech problems such as finding words, apraxia, and articulation problems, sleep regulation problems, eye or muscle movement problems, chronic fatigue problems, digestion problems, sensitivity to chemicals, viral infection, inflammatory conditions such as rheumatoid arthritis, sciatica, and fibromyalgia, asthma, irritable bowel, colitis, tinnitus, migraines, nail biting, and autoimmune problems.

2. The method of claim 1, wherein methylcobalamin is administered.

3. The method of claim 1, wherein methylcobalamin, or a pharmaceutically acceptable salt thereof, is administered with folinic acid or a pharmaceutically acceptable salt of folinic acid.

4. The method of claim 3, wherein methylcobalamin and folinic acid are administered.

5. The method of claim 1, wherein methylcobalamin is administered in a dosage of about 500-1500 μg/0.1 ml of spray, per nostril once per day.

6. The method of claim 5, further comprising administering folinic acid with the methylcobalamin, wherein the folinic acid is administered in a dosage of about 25-300 μg/0.1 ml of spray, per nostril once per day.

7. The method of claim 1, wherein the disorder is ADHD, anxiety, stress and chronic stress, or irritable bowel.

8. A method of treating a psychological or neurophysiological disorder, comprising nasally administering methylcobalamin, or a pharmaceutically acceptable salt thereof, to a person in need of such treatment in an amount sufficient to treat the disorder in the person, wherein the disorder is attention deficit hyperactivity disorder (ADHD), anxiety, stress and chronic stress, or irritable bowel.

9. The method of claim 8, wherein methylcobalmin is administered.

10. The method of claim 8, wherein methylcobalamin, or a pharmaceutically acceptable salt thereof, is administered with folinic acid or a pharmaceutically acceptable salt of folinic acid.

11. The method of claim 10, wherein methylcobalamin and folinic acid are administered.

12. The method of claim 8, wherein methylcobalamin is administered in a dosage of about 500-1500 μg/0.1 ml of spray, per nostril once per day.

13. The method of claim 12, further comprising administering folinic acid with the methylcobalamin, wherein the folinic acid is administered in a dosage of about 25-300 μg/0.1 ml of spray, per nostril once per day.

14. A method of treating attention deficit hyperactivity disorder (ADHD), comprising nasally administering methylcobalamin, or a pharmaceutically acceptable salt thereof, to a person in need of such treatment in an amount sufficient to treat ADHD in the person.

15. The method of claim 14, wherein methylcobalamin is administered.

16. The method of claim 14, wherein methylcobalamin, or a pharmaceutically acceptable salt thereof, is administered with folinic acid or a pharmaceutically acceptable salt of folinic acid.

17. The method of claim 16, wherein methylcobalamin and folinic acid are administered.

18. The method of claim 14, wherein the treatment reduces hyperactivity or inattentiveness symptoms of ADHD.

19. The method of claim 14, wherein methylcobalamin is administered in a dosage of about 500-1500 μg/0.1 ml of spray, per nostril once per day.

20. The method of claim 19, further comprising administering folinic acid with the methylcobalamin, wherein the folinic acid is administered in a dosage of about 25-300 μg/0.1 ml of spray, per nostril once per day.
